(19) United States
(12) Patent Application Publication
(10) Pub. No.: US 2009/0318876 A1
(43) Pub. Date: Dec. 24, 2009

(54) Determination of a Type and an Amount of a Medicament by Inductive Means

(75) Inventors: Jon Ulrich Hansen, Herlev (DK); Lennart Berggren, Lund (SE)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/307,597

(22) PCT Filed: Jun. 27, 2007
(86) PCT No.: PCT/EP07/56418

§ 371 Date: Apr. 21, 2009

(30) Foreign Application Priority Data
Jul. 7, 2006 (EP) 06014117.3

(51) Int. Cl. A61M 5/31; G06K 19/06; G06K 7/08

(52) U.S. Cl. 604/187; 235/492; 235/449

(57) ABSTRACT

The present invention relates to a medication delivery device (1) comprising a cartridge compartment adapted to receive and hold a medicament containing cartridge (4), the cartridge compartment comprising an electromagnetic device (5) formed as an induction coil and adapted to generate a magnetic field having a spatial overlap with at least part of a medicament in a cartridge positioned in the cartridge compartment, the electromagnetic device forming part of an electronic circuit adapted to oscillate at a predetermined resonance frequency. The present invention further relates to a cartridge and a label comprising an electromagnetic device adapted to generate a magnetic field having a spatial overlap with at least part of a medicament in a cartridge.

### FIELD OF THE INVENTION

The present invention relates to various arrangements comprising an electromagnetic device adapted to generate a magnetic field having a spatial overlap with a medicament, such as insulin, contained in a medication delivery device, a medicament containing cartridge or the like. The electromagnetic device forms an integral part of an oscillating circuit having a well-defined resonance frequency and an associated Q-factor. The resonance frequency and Q-factor of the oscillating circuit varies with a dielectric constant being present in the magnetic field of the electromagnetic device. Alternatively, the electromagnetic device may form an integral part of an electronic circuit being capable of providing pulses to the electromagnetic device. In this situation the type of medicament is determined by analyzing a pulse response signal from the electromagnetic device. The present invention further relates to an associated method for distinguishing different types of insulin, and to a method for determining an amount of medicament in a cartridge.

### BACKGROUND OF THE INVENTION

U.S. Pat. No. 5,782,814 discloses an apparatus for determining and recording a dose of an agent delivered with a syringe. The syringe has a barrel for holding the agent and a plunger movably positioned in the barrel for expelling the agent. The plunger includes a magnetically responsive element, such as an iron core. The apparatus suggested in U.S. Pat. No. 5,782,814 has a receptacle for receiving the syringe for dose measurement. An inductive element is positioned coaxially to the receptacle to produce a magnetic field. When the syringe is placed in the receptacle, the strength of the magnetic field varies in dependence upon the position of the plunger in the barrel. Thus, the strength of the magnetic field is indicative of the axial position of the plunger, and therefore indicative of a remaining amount of agent in the syringe. The magnetic field induces a voltage in a conducting loop. A voltage meter is connected to the conducting loop to measure the induced voltage which is indicative of the axial position of the plunger. A microprocessor is operatively connected to the voltage meter in order to calculate the remaining amount of agent from the induced voltage. A recorder, such as a digital memory unit, records the calculated amount of agent.

The apparatus suggested in U.S. Pat. No. 5,782,814 is purely intended for determining an amount of agent in a syringe, or alternative, determining an amount of agent delivered by a syringe.

A paper by Insuk Yu, “Electrodeless measurement of RF dielectric constant and loss” published in Meas. Sci. Technol. 4 (1993) 344-348 discloses a method by which liquids having different dielectric constants can be distinguished. The method of distinguishing liquids having different dielectric constants implies that the liquids are positioned in a magnetic field of a coil which forms part of an oscillating circuit. Upon positioning a sample which in this case is a liquid having a given dielectric constant the oscillating condition and Q-factor of the oscillating circuit is changed. This change depends on the dielectric constant of the liquid positioned in the magnetic field of the coil.

There is in the above-mentioned paper by Insuk Yu no mentioning of an appliance of the method in connection with distinguishing various types of medicament, such as insulin. Thus, there is in the above-mentioned paper no mentioning or hint in a direction of implementing the method in medication delivery devices in general, such as in for example insulin delivery devices.

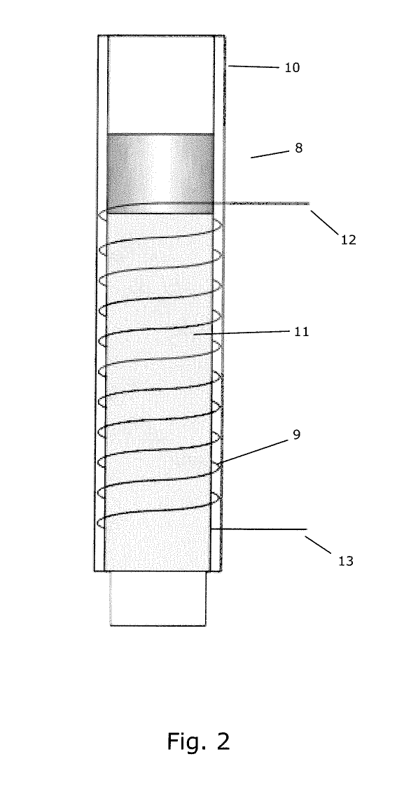

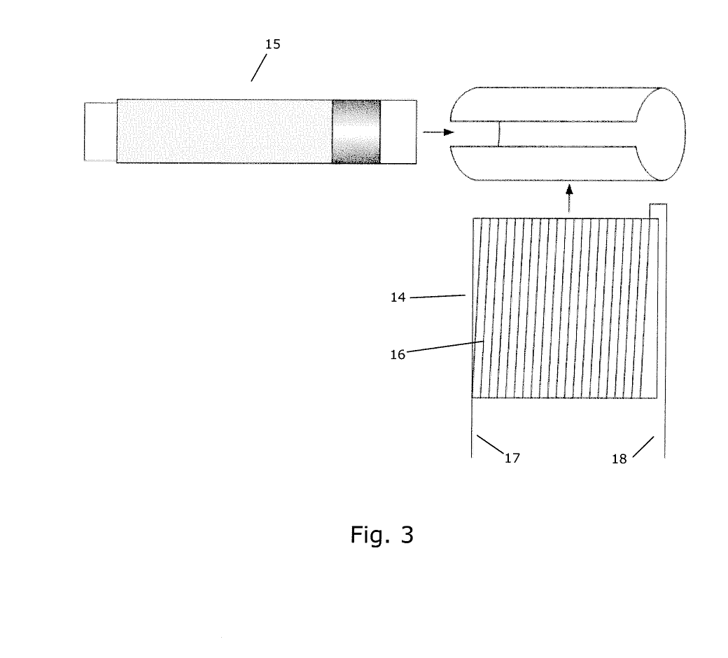

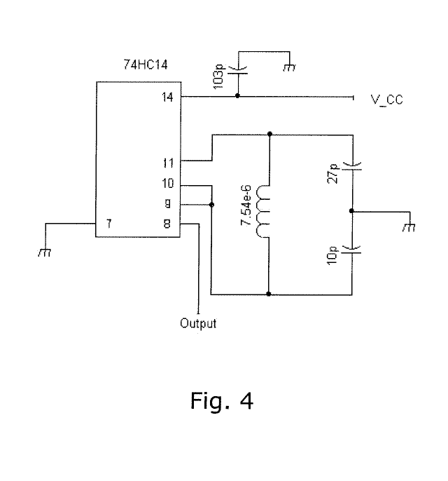

U.S. Pat. No. 6,068,615 discloses various ways of measuring quantities of insulin in traditional syringes. In FIG. 2 of U.S. Pat. No. 6,068,615 a measuring apparatus (120) adapted to receive a traditional syringe (80) is disclosed. An inductor (100) is arranged around the syringe (80) when the syringe (80) is positioned in the measuring apparatus (120). Thus, in order to determine the quantity of insulin in the syringe an, in relation to the syringe, external measuring apparatus (120) is required. FIGS. 3 and 4 of U.S. Pat. No. 6,068,615 are concerned with traditional syringes where insulin is filled into chambers (92) of the syringes. Thus, the syringes (480) and (580) of FIGS. 3 and 4 of U.S. Pat. No. 6,068,615 are certainly not adapted to receive self-contained insulin containing cartridges.

WO 2006/021295 relates to an arrangement for determining the filling level of a substance in an ampoule. The arrangement comprises at least two electrodes between which the substance can be introduced. WO 2006/021295 further relates to a method for determining the filling level of a substance in an ampoule comprising at least two electrodes, whereby the filling level is determined by measuring the capacity of at least one capacitor involving the dielectric constant of the substance and the two electrodes between which the substance is positioned. Thus, WO 2006/021295 relates to an arrangement and a method for determining a substance level or quantity by capacitive means.

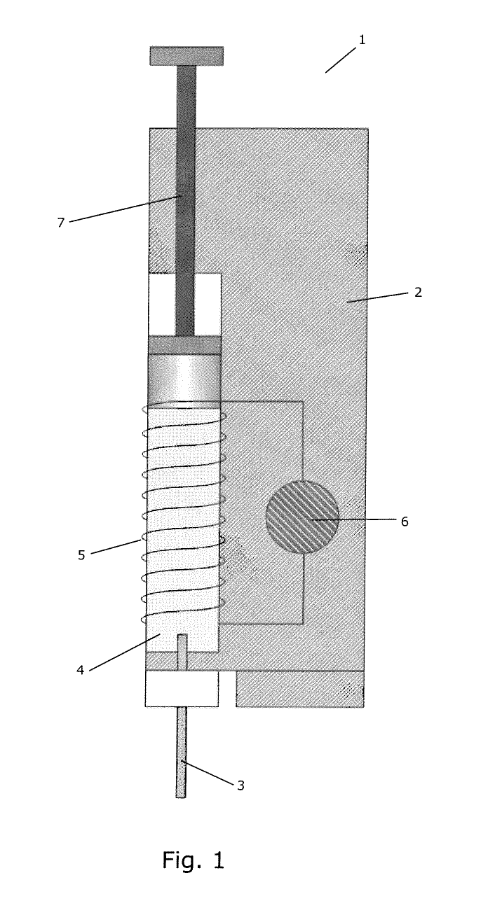

US 2002/188259 relates in general to product authentication by way of identification and certification of the origin or manufacturer of medical supplies, components etc. FIG. 1 of US 2002/188259 shows an RFID tag attached to a thin backing of for example self-adhesive paper. The RFID tag consists of a miniature integrated circuit and an antenna. A conductive loop of for example conductive ink or fine breakable conducting wire such as copper, is deposited on the backing and is in electrical connection to the integrated circuit via conductive traces and pads. The RFID tag allows identification of the medical supply to which the thin backing including the RFID tag is attached.

WO 2005/111961 also relates to an RFID tag attached to a thin backing. Also here the RFID tag involves a miniature integrated circuit electrically coupled to an antenna.

There are in US 2002/188259 and WO 2005/111961 no information as to the generation of magnetic fields for determining a type of medication or an amount of medicament.

WO 01/56635 relates to a substance containing container having a recognition element associated therewith. WO 01/56635 further relates to a sensor element adapted to read information out of or inscribe information into the recognition element. Similar to US 2002/188259 and WO 2005/111961 there is in WO 01/56635 no information as to the generation of magnetic fields for determining a type of medication or an amount of medicament.

It is an object of the present invention to provide an arrangement incorporated into a hand-held medication delivery device, a cartridge or similar device, the arrangement being capable of distinguishing liquids or medicaments, such as insulin, having different dielectric constants.

### SUMMARY OF THE INVENTION

The above-mentioned object is complied with by providing, in a first aspect, a hand-held medication delivery device adapted to expel set doses of medicament into for example the body of a patient, the medication delivery device comprising a cartridge compartment adapted to receive and hold a medicament containing cartridge, the cartridge compartment comprising an electromagnetic device adapted to generate a magnetic field having a spatial overlap with at least part of a medicament in a cartridge positioned in the cartridge compartment, the electromagnetic device forming part of an electronic circuit adapted to oscillate at a predetermined resonance frequency or alternatively, forming part of an electronic circuit being capable of generating and providing pulses to the electromagnetic device. The resonance frequency of the electronic circuit may be controlled by an oscillating crystal or a microprocessor. The medicament containing cartridge may have substantially rigid outer sidewalls or it may have flexible outer sidewalls thus forming a cartridge with varying outer dimensions in combination with a flexible shape.

The medication delivery device according to the first aspect of the present invention may be a manual, a semi-automatic, an automatic or a motor driven medication delivery device for injecting or delivering a medicament, such as insulin, into a patient. By manual medication delivery device is meant a device where the medicament is expelled from the device purely by means of a force provided by the user of the manual medication delivery device. By semi-automatic and automatic is meant that the energy necessary to expel the medicament from the device is at least partly accumulated in the device. As an example such energy may be accumulated or stored in a resilient member, such as in a torsion or axial spring. Finally, the medication delivery device may be a motor-based device where the medicament is expelled from the device by use of an electric motor.

As used herein, the term medicament is meant to encompass any medicament-containing flowable medicine capable of being passed through a delivery means, such as a hollow needle, in a controlled manner. Thus, the term medicament covers liquid solutions, gels or fine suspension. Representative medicaments include pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

The electromagnetic device may comprise a coil of wound wire or alternatively, another electromagnetic device capable of generating a magnetic field. Such other device may be an electrically conductive path arranged, for example, on a printed circuit board, such as a flex-print. In the following the electromagnetic device will be described with reference to a coil of wound wire but it should be understood that the present invention is not limited to such a device. The wound wire may be arranged in such a manner that it forms an essentially helical path around the cartridge when said cartridge is positioned in the cartridge compartment. The essentially helical path of wound wire may define a centre axis, said centre axis essentially coinciding with a centre axis of the cartridge when said cartridge is positioned in the cartridge compartment of the medication delivery device. The length of the essentially helical path in a direction parallel to its centre axis substantially may equal a length of a medicament containing container of the cartridge. The term medicament containing container is here to be understood as that part of the cartridge which is capable of containing a medicament.

The electronic circuit may comprise one or more capacitors adapted to exchange energy with the electromagnetic device.

In a second aspect, the present invention relates to a cartridge for a medication delivery device, the cartridge comprising a medicament containing container comprising an electromagnetic device adapted to generate a magnetic field having a spatial overlap with at least part of a medicament in the medicament containing container, the electromagnetic device being connectable to an electronic circuit adapted to oscillate at a predetermined resonance frequency or alternatively, connectable to an electronic circuit being capable of generating and providing pulses to the electromagnetic device. Again, the resonance frequency of the electronic circuit may be controlled by an oscillating crystal or a microprocessor. The cartridge may have substantially rigid outer sidewalls similar to a traditionally, cylindrically shaped cartridge or it may have flexible outer sidewalls thus forming a cartridge with varying outer dimensions in combination with a flexible shape.

Again, the term medicament containing container is to be understood as that part of the cartridge which is capable of containing a medicament. As with the medication delivery device the electromagnetic device may comprise a coil of wound wire shaped as an essentially helical path. The essentially helical path of wound wire may define a centre axis, said centre axis essentially coinciding with a centre axis of the medicament containing container. The length of the essentially helical path in a direction parallel to its centre axis substantially may equal a length of the medicament containing container.

The wound wire forming the essentially helical path may be embedded into one or more sidewall portions of the medicament containing container. These sidewall portions may be essentially cylindrically shaped. Alternatively, the wound wire forming the essentially helical path may be manufactured separately and thereafter arranged on one or more exterior sidewall portions of the medicament containing container.

The electronic circuit, including one or more capacitors adapted to exchange energy with the electromagnetic device, may form an integral part of the cartridge.

In a third aspect, the present invention relates to a label adapted to be arranged on an exterior surface portion of a medicament containing container of a cartridge for a medication delivery device, the label comprising an integrated electromagnetic device adapted to generate a magnetic field having a spatial overlap with at least part of a medicament in the medicament containing container when the label is arranged on the exterior surface portion of the medicament containing container, the electromagnetic device being connectable to an electronic circuit adapted to oscillate at a predetermined resonance frequency or alternatively, connectable to an electronic circuit being capable of generating and providing pulses to the electromagnetic device. The resonance frequency of the electronic circuit may be controlled by an oscillating crystal or a microprocessor.

The label may be implemented as a flexible, self-adhesive label which is adapted to follow contours of the exterior surface portion of the medicament containing container. The electromagnetic device may form an essentially helical-shaped coil when the label is arranged on the exterior surface portion of the medicament containing container. The essentially helical-shaped coil may define a centre axis, said centre axis essentially coinciding with a centre axis of the medicament containing container when the label is arranged on the exterior surface portion of the medicament containing container. The length of the essentially helical-shaped coil in a direction parallel to its centre axis substantially may equal a length of the medicament containing container.

The electronic circuit may form an integral part of the label. In addition, the electronic circuit may comprise a capacitor adapted to exchange energy with the electromagnetic device.

In a fourth aspect, the present invention relates to a method for identifying a type of medicament in a cartridge for insertion into a cartridge compartment of a medication delivery device, the method comprising the steps of
providing an electronic circuit adapted to oscillate at a first resonance frequency,
positioning the cartridge containing the medicament in a magnetic field generated by the inductor, and determining a second resonance frequency of the electronic circuit, and
determining, by comparing the second resonance frequency to predetermined values in a look-up table, the type of medicament in the cartridge.

In a fifth aspect, the present invention relates to a method for determining an amount of medicament in a cartridge for insertion into a cartridge compartment of a medication delivery device, the method comprising the steps of
providing an electronic circuit adapted to oscillate at a first resonance frequency,
positioning the cartridge containing the medicament in a magnetic field generated by the inductor, and determining a second resonance frequency of the electronic circuit, and
determining, by comparing the second resonance frequency to predetermined values in a look-up table, the amount of medicament in the cartridge.

Thus, according to the fourth and fifth aspects of the present invention a type and an amount of a medicament in a cartridge may be determined from a modified resonance frequency by comparing this modified resonance frequency with pre-stored frequency values, such as frequency values of an electronic look-up table.

In case the modified resonance frequency matches, within pre-specified tolerances, a value in the look-up table the type of medicament in the cartridge may be unambiguously determined in that each value of the look-up table is associated with a given type of medicament in a completely full cartridge. However, the type of medicament in the cartridge may also be unambiguously determined in case the cartridge is only partly filled. When the type of medicament has been determined measurements of the modified resonance frequency may be applied to determine a remaining amount of medicament in the cartridge. If a match between a modified resonance frequency and the look-up table, within pre-specified tolerances, is reached the remaining amount of medicament in the cartridge may be unambiguously determined.

The electronic circuit of the fourth and fifth aspects may comprise an inductor operatively coupled to a capacitor. Alternatively, the resonance frequency of the electronic circuit may be controlled by an oscillating crystal or a microprocessor.

In a sixth aspect, the present invention relates to a method for identifying a type of medicament in a cartridge for insertion into a cartridge compartment of a medication delivery device, the method comprising the steps of
providing an electronic circuit adapted to generate and provide one or more electric pulses to an inductor of the electronic circuit,
positioning the cartridge containing the medicament in a magnetic field generated by the inductor, and determining a pulse response signal from the inductor, and
determining, by comparing the pulse response signal to predetermined values in a look-up table, the type of medicament in the cartridge.

In a seventh aspect, the present invention relates to a method for determining an amount of medicament in a cartridge for insertion into a cartridge compartment of a medication delivery device, the method comprising the steps of
providing an electronic circuit adapted to generate and provide one or more electric pulses to an inductor of the electronic circuit,
positioning the cartridge containing the medicament in a magnetic field generated by the inductor, and determining a pulse response signal from the inductor, and
determining, by comparing the pulse response signal to predetermined values in a look-up table, the amount of medicament in the cartridge.

### BRIEF DESCRIPTION OF THE INVENTION

The present invention will now be explained in further details with reference to the accompanying figures, wherein

FIG. 1 shows a medication delivery device having a coil positioned in the cartridge compartment,

FIG. 2 shows a medicament containing cartridge having a coil integrated therewith,

FIG. 3 shows a label adapted to be positioned on a medicament containing cartridge, and

FIG. 4 shows a circuit diagram of a first oscillator,

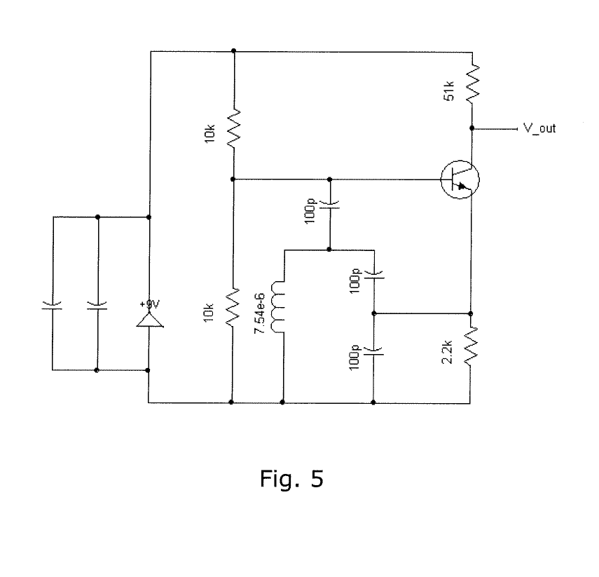

FIG. 5 shows a circuit diagram of a second oscillator,

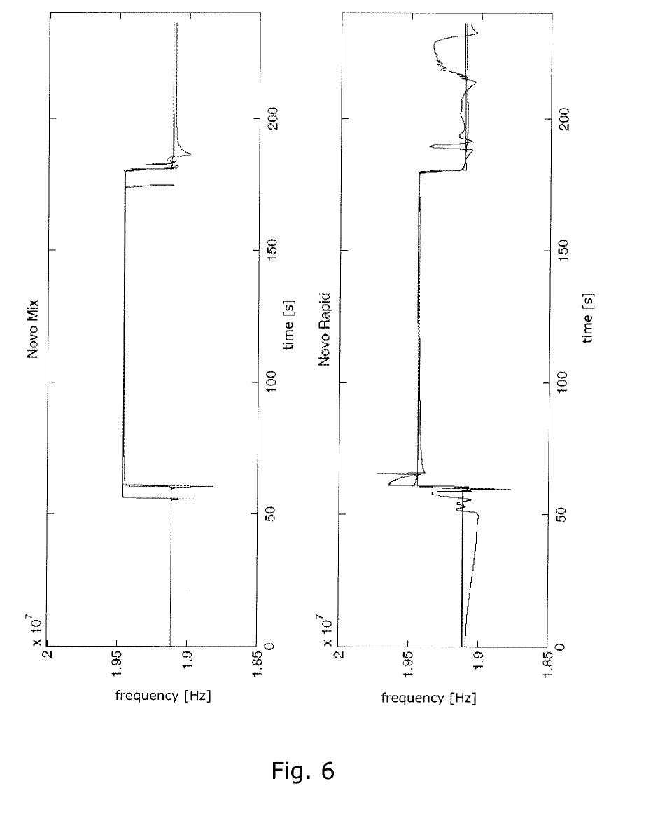

FIG. 6 shows the frequency shift when two different samples (Novo Mix & Novo Rapid) are positioned in the inductor,

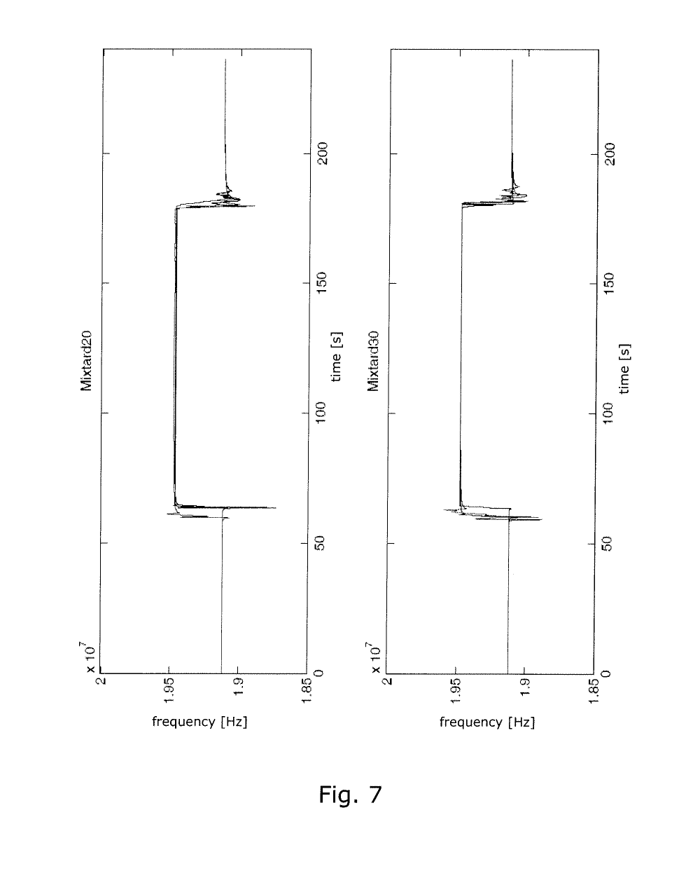

FIG. 7 shows the frequency shift when two different samples (Mixtrad20 & Mixtrad30) are positioned in the inductor,

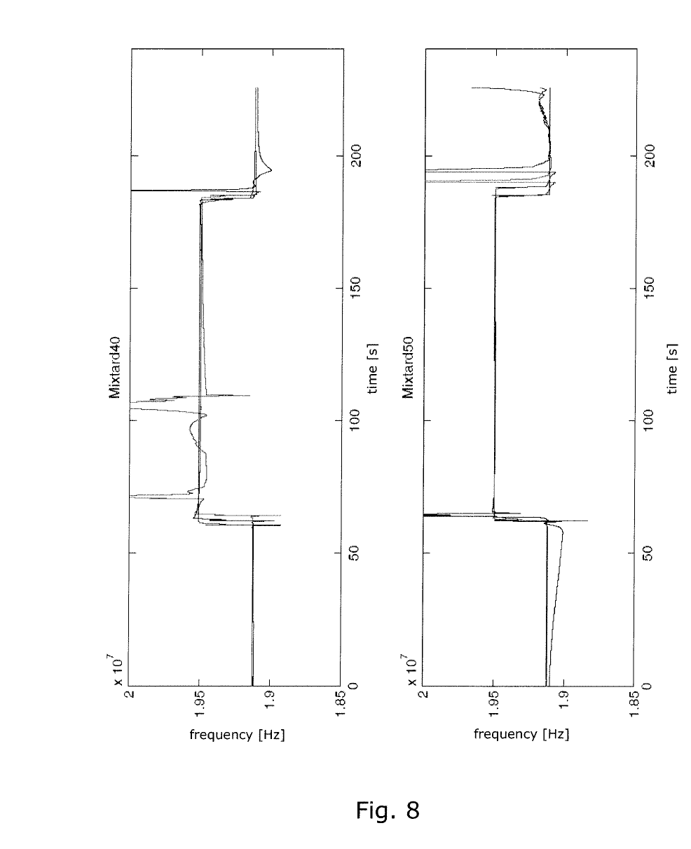

FIG. 8 shows the frequency shift when two different samples (Mixtrad40 & Mixtrad50) are positioned in the inductor,

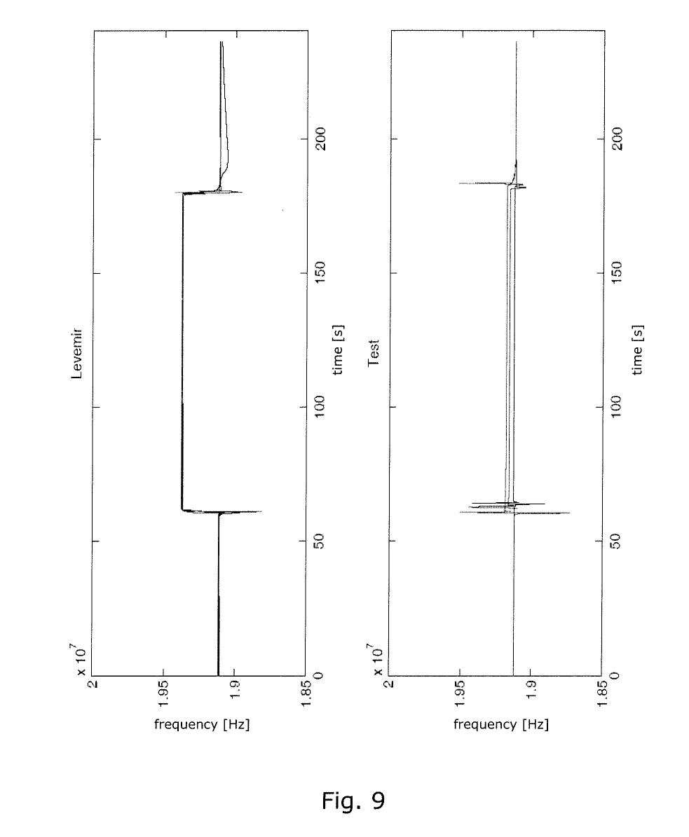

FIG. 9 shows the frequency shift when two different samples (Levemir & Test) are positioned in the inductor,

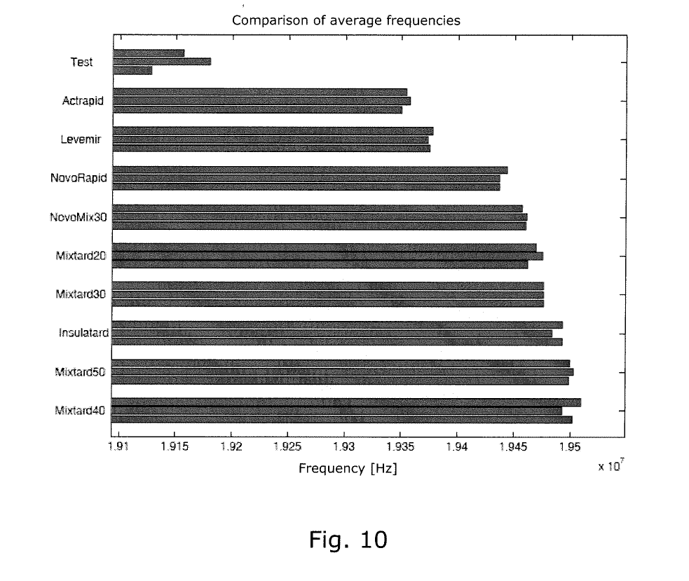

FIG. 10 shows a comparison of average frequencies of different samples positioned in the inductor of the first oscillator,

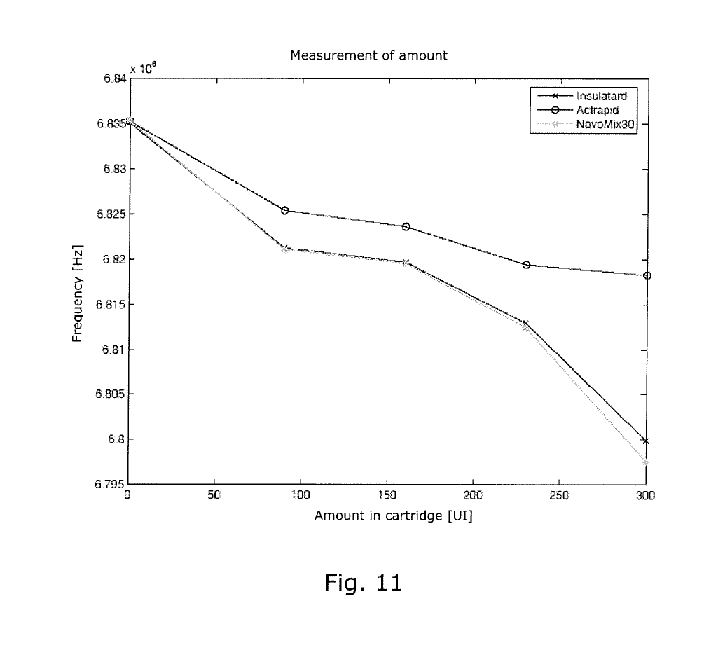

FIG. 11 shows the resonance frequency as a function of the volume of the sample in the cartridge, and

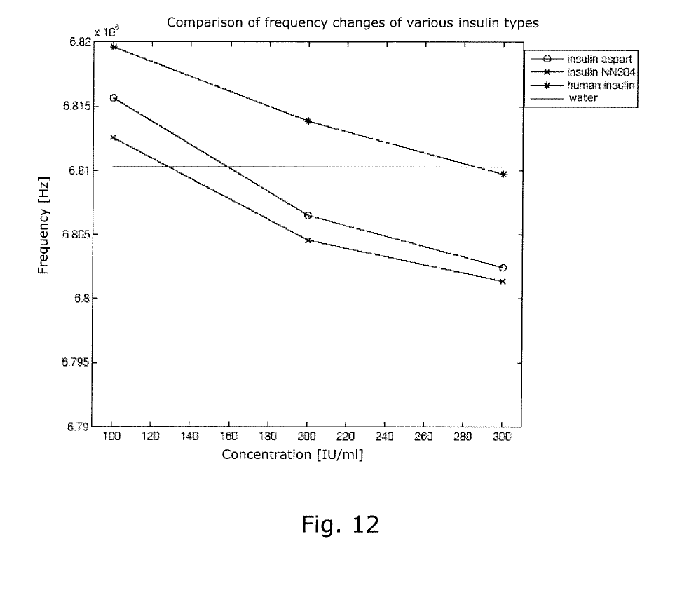

FIG. 12 shows the resonance frequency as a function of insulin concentration in the cartridge.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

In its most general aspect the present invention relates to an arrangement for contact free determination of a type of medicament in a cartridge. Thus, by incorporating the arrangement according to the present invention in a cartridge compartment of a medication delivery device, or by incorporating the arrangement according to the present invention in a medicament containing cartridge various types of medicaments can be distinguished from each other. Thus, in case of an insulin delivery device the type of insulin present in the device can be determined and thereby communicated to the patient using the insulin delivery device. In fact, an insulin delivery device may be pre-programmed only to accept certain types of insulin. In case a wrong type of insulin is inserted into the insulin delivery device an alarm signal may be generated informing the patient that he or she has inserted a wrong type of insulin in the device. In addition, means for preventing the insulin delivery device from being operable in case a wrong type of insulin is inserted into the device can be provided.

FIG. 1 shows a medication delivery 1 device comprising a helical-shaped coil in the cartridge compartment. The medication delivery device 1 comprises a housing 2, an injection needle 3, a cartridge 4, a helical-shaped inductor 5 and an oscillating circuit 6 operatively connected to the inductor 5. The medication delivery device 1 further comprises a displaceable piston rod 7 which, when displaced towards the injection needle 3, expels medicament, such as insulin, from the cartridge 4.

As already mentioned the medication delivery device can be a manual, a semi-automatic, an automatic or a motor driven medication delivery device for injecting or delivering a medicament, such as insulin, into a patient. By manual medication delivery device is meant a device where the medicament is expelled from the device purely by means of a force provided by the user of the manual medication delivery device. By semi-automatic and automatic is meant that the energy necessary to expel the medicament from the device is at least part accumulated in the device. As an example such energy may be accumulated or stored in a resilient member, such as in a torsion or axial spring. Finally, the medication delivery device may be a motor-based device where the medicament is expelled from the device by use of an electric motor.

The helical-shaped inductor 5 generates a magnetic field in the axial direction of the cartridge 4. In principle, the inductor 5 can also be oriented differently, i.e. generating a magnetic field in a different direction relative to the cartridge.

The resonance frequency of the oscillating circuit may in principle be chosen arbitrarily. In the present invention a so-called Colpitts-oscillator oscillating at a frequency around 19 MHz and a more frequency stable oscillator oscillating a frequency around 7 MHz have been applied. Circuit diagrams of these oscillators are shown in FIGS. 4 and 5. However, other resonance frequencies and other circuit diagrams are also applicable.

FIG. 2 shows a medicament containing cartridge 8 comprising an integrated helical-shaped coil 9. As seen in FIG. 2 the helical-shaped coil 9 is integrated in the cylindrically shaped sidewall portions 10 and circumvents, in the axial direction of the cylindrically shaped cartridge, a majority of medicament 11 contained in the cartridge. The free ends 12, 13 of the coil are adapted to be connected to an oscillator of the kind depicted in FIGS. 4 and 5. The oscillator may be positioned in an associated medication delivery device into which the cartridge is adapted to be inserted. Alternatively, the oscillator can be integrated with the cartridge so that only supply power and data relating to the medicament in the cartridge is provided across the medication delivery device/cartridge interface.

FIG. 3 shows a self-adhesive label 14 adapted to position on an exterior surface portion of a medicament containing cartridge 15. The self-adhesive label comprises an integrated electrically conductive path 16 which forms an essentially helical-shaped coil when the label 14 is positioned on the exterior surface portion of the medicament containing cartridge 15. Again, the free ends 17, 18 of the electrically conducting path are adapted to be connected to an oscillator of the kind depicted in FIGS. 4 and 5. The oscillator may be positioned in an associated medication delivery device into which the cartridge with the label attached thereto is adapted to be inserted. Alternatively, the oscillator can be integrated with the label or the cartridge so that only supply power and data relating to the medicament in the cartridge is provided across the medication delivery device/cartridge interface.

FIGS. 4 and 5 show examples of electrical circuits, in the following denoted oscillators, being capable of oscillating at predetermined resonance frequencies. The oscillator depicted in FIG. 4 is the so-called Colpitts-oscillator which oscillates at a frequency around 19 MHz. The inductor into which a medicament is inserted has an inductance of around 7.5 μH. FIG. 5 shows a more frequency stable oscillator. This oscillator oscillates at a frequency around 7 MHz.

The oscillator can be arranged in various manners. For example, the complete circuit including the inductor can be positioned within the medication delivery device. However, the oscillator can also be split between the medicament containing cartridge and the medicament delivery device in that the inductor can be arranged on or integrated with the cartridge whereas the rest of the oscillator is arranged within the medication delivery device. Obviously, an electrical connection between the inductor and the rest of the oscillator must be provided. Alternative, the complete oscillator can be arranged on or integrated with the medicament containing cartridge.

It should be noted that other principles than shifts in resonance frequencies may be applied to measure characteristics of a medicament positioned in a magnetic field. For example, an electronic circuit adapted to generate and provide one or more electric pulses to the inductor is also applicable. In this situation the type of medicament will be determined by analyzing the pulse response signal from the inductor when the medicament is positioned in the inductor and one or more electric pulses are provided to the inductor.

FIGS. 6-9 illustrate how the resonance frequency shifts when cartridges containing various medicaments are positioned in the inductor of the oscillator depicted in FIG. 4. The medicaments used for demonstrating the frequency shift are all insulin products from Novo Nordisk. The products are as follows: NovoMix30, Novo Rapid, Mixtard20, Mixtard30, Mixtard40, Mixtard50, Levemir and a test sample. The samples were inserted into the inductor after around 60 s and removed again after around 180 s.

As seen in FIGS. 6-8 the resonance frequency is shifted approximately 450 kHz when the medicaments are inserted into the inductor. After the medicaments have been removed the resonance frequency returns to its initial value, i.e. its value before the medicaments were inserted into the inductor. FIG. 9 shows that the medicament Levemir also shifts the resonance frequency by around 450 kHz whereas the test sample (liquid without insulin) induces only a minor frequency shift of the resonance frequency.

FIG. 10 shows a comparison between average resonance frequencies induced by samples of medicament positioned in the inductor of the oscillator depicted in FIG. 4. As seen, the average resonance frequencies are different for most of the medicaments which means that the chance of the medicaments being distinguishable by their respective resonance frequencies only is reasonable.

FIG. 11 shows the resonance frequency of the oscillator shown in FIG. 5 as a function of the volumes of three different medicaments, Insulatard, Actrapid and NovoMix30. As seen, the higher the volume the lower the resonance frequency. This tendency applies to all three medicaments. As the volume approaches zero the resonance frequency approaches the oscillating frequency of the oscillator, i.e. 6.84 MHz.

FIG. 12 shows the resonance frequency of the oscillator shown in FIG. 5 as a function of the concentration of three types of insulin; insulin aspart, insulin NN304 and human insulin. The horizontal line indicates the oscillating frequency in case water is positioned in the inductor of the oscillator shown in FIG. 5. The lines representing the measurements on the three types of insulin show a clear tendency towards lower resonance frequency with higher concentration of insulin.

1. A hand-held medication delivery device adapted to expel set doses of medicament, the medication delivery device comprising:
a cartridge compartment adapted to receive and hold a medicament containing cartridge, the cartridge compartment comprising an electromagnetic device adapted to generate a magnetic field having a spatial overlap with at least part of a medicament in a cartridge positioned in the cartridge compartment.

2. A medication delivery device according to claim 1, wherein the electromagnetic device forms part of an electronic circuit adapted to oscillate at a predetermined resonance frequency.

3. A medication delivery device according to claim 1, wherein the electromagnetic device forms part of an electronic circuit adapted to generate and provide one or more pulses to the electromagnetic device.

4. A medication delivery device according to claim 1, wherein the electromagnetic device comprises a coil of wound wire.

5. A medication delivery device according to claim 4, wherein the coil of wound wire forms an essentially helical path.

6. A medication delivery device according to claim 5, wherein an essentially helical path of wound wire defines a centre axis, said centre axis essentially coinciding with a centre axis of the cartridge when said cartridge is positioned in the cartridge compartment of the medication delivery device.

7. A medication delivery device according to claim 6, wherein the length of the essentially helical path in a direction parallel to its centre axis substantially equals a length of a medicament containing container of the cartridge.

8. A medication delivery device according to claim 2, wherein the electronic circuit comprises a capacitor adapted to exchange energy with the electromagnetic device.

9. A medication delivery device according to claim 2, wherein the electronic circuit comprises an oscillating crystal or a microprocessor.

10. A cartridge for a medication delivery device, the cartridge comprising:
a medicament containing container comprising an electromagnetic device adapted to generate a magnetic field having a spatial overlap with at least part of a medicament in the medicament containing container, the electromagnetic device being connectable to an electronic circuit.

11. A cartridge according to claim 10, wherein the electronic circuit is adapted to oscillate at a predetermined resonance frequency.

12. A cartridge according to claim 10, wherein the electronic circuit is adapted to generate and provide one or more pulses to the electromagnetic device.

13. A cartridge according to claim 10, wherein the electromagnetic device comprises a coil of wound wire.

14. A cartridge according to claim 13, wherein the coil of wound wire form an essentially helical path.

15. A cartridge according to claim 14, wherein the essentially helical path of wound wire defines a centre axis, said centre axis essentially coinciding with a centre axis of the medicament containing container.

16. A cartridge according to claim 15, wherein the length of the essentially helical path in a direction parallel to its centre axis substantially equals a length of the medicament containing container.

17. A cartridge according to claim 16, wherein the wound wire forming the essentially helical path is embedded into one or more sidewall portions of the medicament containing container.

18. A cartridge according to claim 16, wherein the wound wire forming the essentially helical path is arranged on one or more exterior sidewall portions of the medicament containing container.

19. A cartridge according to claim 10, wherein the electronic circuit form an integral part of the cartridge.

20. A cartridge according to claim 19, wherein the electronic circuit comprises a capacitor adapted to exchange energy with the electromagnetic device.

21. A label adapted to be arranged on an exterior surface portion of a medicament containing container of a cartridge for a medication delivery device, the label comprising:
an integrated electromagnetic device adapted to generate a magnetic field having a spatial overlap with at least part of a medicament in the medicament containing container when the label is arranged on the exterior surface portion of the medicament containing container, and
the electromagnetic device being connectable to an electronic circuit.

22. A label according to claim 21, wherein the electronic circuit is adapted to oscillate at a predetermined resonance frequency.

23. A label according to claim 21, wherein the electronic circuit is adapted to generate and provide one or more pulses to the electromagnetic device.

24. A label according to claim 21, wherein the label comprises a flexible, self-adhesive label which is adapted to follow contours of the exterior surface portion of the medicament containing container.

25. A label according to claim 21, wherein the electromagnetic device forms an essentially helical-shaped coil when the label is arranged on the exterior surface portion of the medicament containing container.

26. A label according to claim 25, wherein the essentially helical-shaped coil defines a centre axis, said centre axis essentially coinciding with a centre axis of the medicament containing container when the label is arranged on the exterior surface portion of the medicament containing container.

27. A label according to claim 26, wherein the length of the essentially helical-shaped coil in a direction parallel to its centre axis substantially equals a length of the medicament containing container.

28. A label according to claim 21, wherein the electronic circuit form an integral part of the label.

29. A label according to claim 28, wherein the electronic circuit comprises a capacitor adapted to exchange energy with the electromagnetic device.

30. A method for identifying a type of medicament in a cartridge for insertion into a cartridge compartment of a medication delivery device, the method comprising:
providing an electronic circuit adapted to oscillate at a first resonance frequency,
positioning the cartridge containing the medicament in a magnetic field generated by the inductor, and determining a second resonance frequency of the electronic circuit, and
determining, by comparing the second resonance frequency to predetermined values in a look-up table, the type of medicament in the cartridge.

31. A method for determining an amount of medicament in a cartridge for insertion into a cartridge compartment of a medication delivery device, the method comprising:
providing an electronic circuit adapted to oscillate at a first resonance frequency,
positioning the cartridge containing the medicament in a magnetic field generated by the inductor, and determining a second resonance frequency of the electronic circuit, and
determining, by comparing the second resonance frequency to predetermined values in a look-up table, the amount of medicament in the cartridge.

32. A method according to claim 30, wherein the electronic circuit comprises an inductor operatively coupled to a capacitor.

33. A method according to claim 30, wherein the first resonance frequency of the electronic circuit may be controlled by an oscillating crystal or a microprocessor.

34. A method for identifying a type of medicament in a cartridge for insertion into a cartridge compartment of a medication delivery device, the method comprising:
providing an electronic circuit adapted to generate and provide one or more electric pulses to an inductor of the electronic circuit,
positioning the cartridge containing the medicament in a magnetic field generated by the inductor, and determining a pulse response signal from the inductor, and
determining, by comparing the pulse response signal to predetermined values in a look-up table, the type of medicament in the cartridge.

35. A method for determining an amount of medicament in a cartridge for insertion into a cartridge compartment of a medication delivery device, the method comprising:
providing an electronic circuit adapted to generate and provide one or more electric pulses to an inductor of the electronic circuit,
positioning the cartridge containing the medicament in a magnetic field generated by the inductor, and determining a pulse response signal from the inductor, and
determining, by comparing the pulse response signal to predetermined values in a look-up table, the amount of medicament in the cartridge.
